(19) United States
(12) Patent Application Publication
(10) Pub. No.: US 2026/0207181 A1
(43) Pub. Date: Jul. 23, 2026

(54) Women's Urine Collection Cup

(71) Applicant: Darshie Owens, Indianapolis, IN (US)

(72) Inventor: Darshie Owens, Indianapolis, IN (US)

(21) Appl. No.: 19/453,462

(22) Filed: Jan. 20, 2026

Related U.S. Application Data
(60) Provisional application No. 63/748,517, filed on Jan. 23, 2025.

(51) Int. Cl. A61B 10/00

(52) U.S. Cl. CPC A61B 10/007

(57) ABSTRACT

A urine specimen collection cup is described. The collection cup is designed to allow for the easier, more sanitary collection of a urine sample, particularly by women.

### CROSS-REFERENCE TO PRIOR APPLICATIONS

The present application claims priority U.S. Patent Application 63/748,517, filed 23 JAN 2025, currently pending, which is incorporated herein in its entirety by reference.

### FEDERALLY SPONSORED RESEARCH AND DEVELOPMENT

Not applicable.

### JOINT RESEARCH AGREEMENT

Not applicable.

### REFERENCE TO A “SEQUENCE LISTING”, A TABLE, OR A COMPUTER PROGRAM LISTING APPENDIX SUBMITTED ON A COMPACT DISC AND AN INCORPORATION BY REFERENCE OF THE MATERIAL ON THE COMPACT DISC

Not applicable.

### STATEMENT REGARDING PRIOR DISCLOSURES BY AN INVENTOR OR JOINT INVENTOR

Not applicable.

### BACKGROUND OF THE INVENTION

### (1) Field of the Invention

The presently disclosed subject matter relates to a cup for collecting urine specimens, particularly from women.

### (2) Description of Related Art Including Information Disclosed Under 37 CFR 1.97 and 37 CFR 1.98

Many adverse health conditions can be detected by analyzing urine samples from patients. Therefore, it is not uncommon for patients to be asked to collect a urine specimen during a visit to a physician’s office or other healthcare screening facility. This requires the patient to strategically position a small cup to collect a stream of urine.

For women, this can be difficult because the precise location of the urine stream can be irregular and it is necessary to position the cup without the benefit of seeing the urine stream. This can be even more challenging for a pregnant patient because the expansion of the abdominal area can obscure the patient’s view of the urine stream. It is not uncommon for some of the urine to soil the exterior surface of the cup and/or the patient’s hands during collection, making it even more difficult to securely hold the cup to collect the required specimen volume.

Thus, it would be beneficial to have a urine specimen collection cup that is designed to make collection of the urine specimen easier and more sanitary, particularly for women.

### SUMMARY OF THE PRESENT INVENTION

The present development is a urine specimen collection cup. The cup has a body that is a relatively narrow tube shape with ridges on the exterior wall. The cup is sealed at a bottom end, and has a relatively wide oval or circular opening at a top end. The shape of the top end provides a wider opening for collection of urine, particularly by women. The ridges on the body’s exterior reduces the risk of slipperiness if any urine leaks onto the exterior of the cup. Optionally, the ridges may be positioned along the exterior to be consistent with specific liquid volumes within the cup.

### BRIEF DESCRIPTION OF THE FIGURES

FIG. 1 is a perspective view of a first embodiment of a urine specimen collection cup made according to the invention wherein the cup has an oval opening.

FIG. 2 is a perspective view of a second embodiment of a urine specimen collection cup made according to the invention wherein the cup has a circular opening.

### DETAILED DESCRIPTION OF THE PRESENT DEVELOPMENT

The details of one or more embodiments of the presently disclosed subject matter are set forth in this document. Modifications to embodiments described in this document, and other embodiments, will be evident to those of ordinary skill in the art after a study of the information provided in this document. The information provided in this document, and particularly the specific details of the described exemplary embodiments, is provided primarily for clearness of understanding and no unnecessary limitations are to be understood therefrom. In case of conflict, the specification of this document, including definitions, will control.

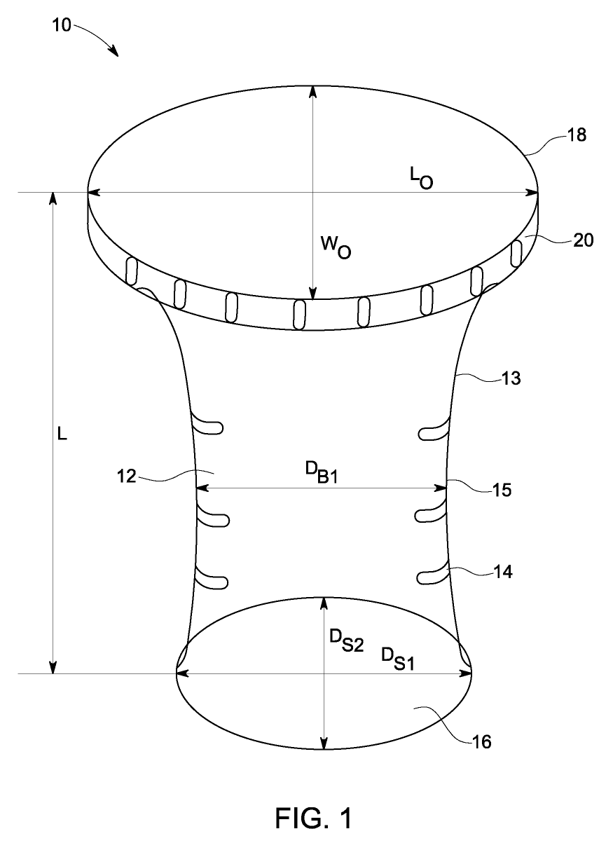

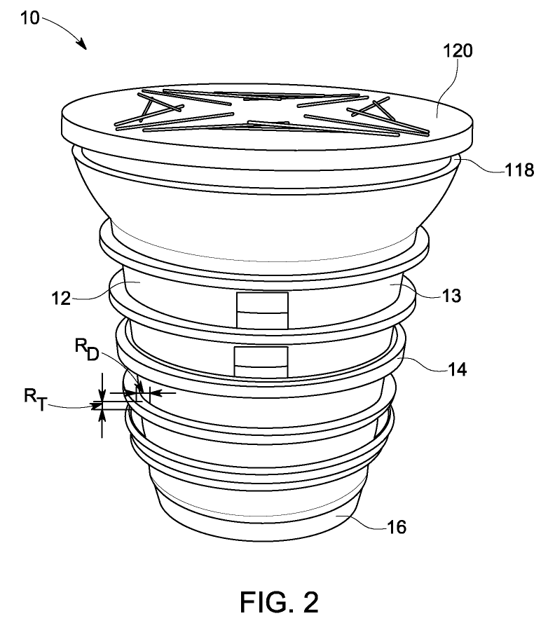

The present development is a urine specimen collection cup. As shown in FIG. 1 and FIG. 2, the cup 10 has a body 12 that is a tube shape and has an exterior wall 13. Ridges 14 extend outwardly from the exterior wall 13. The body 12 has a closed or sealed bottom end 16, so urine cannot flow through the body 12 and exit the cup 10 through the bottom. In an exemplary embodiment, the bottom end 16 may have a circular configuration or an oval configuration.

The body 12 has an opened top end 18. In a first embodiment, as shown in FIG. 1, the top end 18 has an elongated oval configuration. In a second embodiment, as shown in FIG. 2, the top end 118 has a circular configuration. Optionally, the cup 10 may further comprise a removable cap 20, 120 that has the same configuration as the top end 18, 118 and that is proportioned to fit snuggly on the top end 18, 118.

The ridges 14 on the body’s exterior wall 13 reduce the risk of slipperiness if any urine leaks onto the exterior of the cup. Optionally, the ridges 14 may be positioned along the exterior wall 13 to be consistent with specific liquid volumes within the cup.

The urine specimen collection cup 10 has a body with an oval midsection 15, wherein the midsection is defined as the cut through the plane parallel to the bottom end 16 that is located essentially half the distance from the top end 18 to the bottom end 16. The body 12 defines a length, L, and a first body length, D_{B1}, and a second body length, D_{B2}, at the midsection. For a circular configuration midsection, the first body length D_{B1} is essentially equal to the second body length D_{B2}; for an oval configuration midsection, the first body length D_{B1} is greater than the second body length D_{B2}. The bottom sealed end 16 defines a first seal length, D_{S1}, and a second seal length, D_{S2}. For a circular configuration midsection, the first seal length D_{S1} is essentially equal to the second seal length D_{S2}; for an oval configuration midsection, the first seal length D_{S1} is greater than the second seal length D_{S2}. The top opening 18 defines a length, L_{O}, and a width, W_{O}. For an oval configuration top opening 18, the length L_{O} is greater than the width W_{O}; for a circular configuration top opening 118, the length L_{O} is essentially equal to the width W_{O}.

Although not required to function as intended, it is recommended that the bottom sealed end 16 have a configuration similar to the configuration of the midsection 15 with dimensions approximately equal to or slightly larger than the midsection dimensions. In an exemplary embodiment, the body length L is from about 60 millimeters to about 120 millimeters, and more preferably from about 75 millimeters to about 105 millimeters, the body midsection dimensions D_{B1}, D_{B2}.are from about 40 millimeters to about 80 millimeters, and more preferably from about 40 millimeters to about 70 millimeters, the top opening has a length L_{O} of from about 60 millimeters to about 120 millimeters and a width W_{O} of from about 55 millimeters to about 85 millimeters, and the bottom seal dimensions D_{S1}, D_{S2} .are from about 40 millimeters to about 80 millimeters.

Examples: The following examples are intended to provide the reader with a better understanding of the invention. The examples are not intended to be limiting with respect to any element not otherwise limited within the claims

Example 1: A urine specimen collection cup having an oval top opening, a circular body midsection, and a circular bottom seal has a body that measures between about 100 mm long (L). The body midsection has a diameter (D_{B1} = D_{B2}) of about 50 mm, as measured approximately 50 mm from the top opening and approximately 50 mm from the bottom seal. The top opening length (L_{O}) is about 115 mm and width (W_{O}) is about 70 mm. The bottom end seal has a diameter (D_{S1} = D_{S2}) of about 50 mm.

Example 2: A urine specimen collection cup having an oval top opening, an oval body midsection, and an oval bottom seal has a body that measures between about 100 mm long (L). The body midsection has a length D_{B1} of about 55 mm and a width D_{B2} of about 40 mm, as measured approximately 50 mm from the top opening and approximately 50 mm from the bottom seal. The top opening length (L_{O}) is about 100 mm and width (W_{O}) is about 60 mm. The bottom end seal has a length D_{S1} of about 60 mm and a width D_{S2} of about 45 mm.

Example 3: A urine specimen collection cup having a circular top opening, a circular body midsection, and a circular bottom seal has a body that measures between about 80 mm long (L). The body midsection has a diameter (D_{B1} = D_{B2}) of about 65 mm, as measured approximately 40 mm from the top opening and approximately 40 mm from the bottom seal. The top opening length L_{O} is essentially equal to the width W O and is about 80 mm. The bottom end seal has a diameter (D_{S1} = D_{S2}) of about 52 mm.

The ridges 14 may be formed from a plurality of individual bands extending outward from the exterior wall 13 of the cup 10 or from a single band threaded around the exterior wall 13 of the cup 10 . When the ridges are formed from multiple bands, the bands should be positioned essentially parallel to each other with approximately equal spacing between the bands. When the ridges are formed by a single band threaded around the exterior of the cup, the band should be threaded so as to leave approximately equal distance between neighboring ridges. It is recommended, but not required, that each ridge be separated from it immediately neighboring ridges by from about 8 millimeters to about 20 millimeters. It is recommended, but not required, that the ridges have approximately equal depth R_{D} and approximately equal thickness R_{T}. It is recommended, but not required, that the ridge depth R_{D} be from about 1.0 millimeters to about 4.0 millimeters and more preferably from about 2.5 millimeters to about 3.5 millimeters, and that the ridge thickness R_{T} be from about 1.5 millimeters to about 4.0 millimeters and more preferably from about 2.0 millimeters to about 3.0 millimeters,.

The shape of the top end opening of the collection cup provides a wider opening for collection of urine, particularly by women. The ridges on the body’s exterior reduces the risk of slipperiness if any urine leaks onto the exterior of the cup. Thus, the overall design makes collection of the urine specimen easier and more sanitary.

Unless defined otherwise, all technical and scientific terms used herein have the same meaning as commonly understood by one of ordinary skill in the art to which the presently disclosed subject matter pertains. All patents, patent applications, published applications and publications, databases, websites and other published materials referred to throughout the entire disclosure herein, unless noted otherwise, are incorporated by reference in their entirety. Representative methods, devices, and materials are described herein, but are not intended to be limiting unless so noted.

The terms “a”, “an”, and “the” refer to “one or more” when used in the subject specification, including the claims. Unless otherwise indicated, all numbers expressing quantities of components, conditions, and otherwise used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

It is understood that, in light of a reading of the foregoing description, those with ordinary skill in the art will be able to make changes and modifications to the present invention without departing from the spirit or scope of the invention, as defined herein. For example, those skilled in the art may substitute materials supplied by different manufacturers than specified herein without altering the scope of the present invention.

1. A urine specimen collection cup comprising a body defining a tubular structure, wherein the body has one open end and one sealed end, and wherein the body defines an exterior wall, and wherein a plurality of parallel ridges extend from the exterior wall.

2. The urine specimen collection cup of claim 1 wherein the open end has a circular configuration and the sealed end has a circular configuration.

3. The urine specimen collection cup of claim 1 wherein the open end has an oval configuration and the sealed end has a circular configuration.

4. The urine specimen collection cup of claim 1 wherein the open end has an oval configuration and the sealed end has an oval configuration.

5. The urine specimen collection cup of claim 1 wherein the ridges are equidistant from neighboring ridges.

6. The urine specimen collection cup of claim 1 wherein the ridges comprise a plurality of bands oriented parallel to each other.

7. The urine specimen collection cup of claim 1 wherein the ridges comprise a single band threaded around the exterior wall wherein the band is threaded parallel to itself with an equal distance between each thread.

8. The urine specimen collection cup of claim 1 further comprising a cap.
